(19) United States
(12) Patent Application Publication
(10) Pub. No.: US 2008/0139633 A1
(43) Pub. Date: Jun. 12, 2008

(54) CRYSTAL FORM OF 2-(3-FLUORO-4-HYDROXYPHENYL)-7-VINYL-1,3-BENZOXAZOL-5-OL

(75) Inventors: Abdolsamad Tadayon, Kirkland (CA); Silvio Iera, Montreal (CA); Mahmoud Mirmehrabi, Laval (CA)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 11/942,897

(22) Filed: Nov. 20, 2007

Related U.S. Application Data
(60) Provisional application No. 60/860,248, filed on Nov. 21, 2006.

(51) Int. Cl. A61K 31/423; C07D 263/56; A61P 15/00; A61P 25/00; A61P 9/00; A61P 1/00; A61P 35/00; A61P 19/02

(52) U.S. Cl. 514/375; 548/224

(57) ABSTRACT

The present invention is directed to a hydrate crystal form (designated as Form F herein) of 2-(3-fluoro-4-hydroxyphenyl)-7-vinyl-1,3-benzoxazol-5-ol, an estrogenic receptor modulator useful in the treatment of, for example, diseases related to abnormal levels of estrogen.

This application claims benefit of priority to U.S. provisional patent application Ser. No. 60/860,248 filed Nov. 21, 2006, which is hereby incorporated by reference in its entirety.

### FIELD OF THE INVENTION

The present invention is directed to a crystal form (designated as Form F herein) of 2-(3-fluoro-4-hydroxyphenyl)-7-vinyl-1,3-benzoxazol-5-ol, an estrogenic receptor modulator useful in the treatment of, for example, diseases related to abnormal levels of estrogen.

### BACKGROUND OF THE INVENTION

The pleiotropic effects of estrogens in mammalian tissues have been well documented, and it is now appreciated that estrogens affect many organ systems. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences. A class of “coregulatory” proteins can also interact with the ligand-bound receptor and further modulate its transcriptional activity. It has also been shown that estrogen receptors can suppress NF.kappa.B-mediated transcription in both a ligand-dependent and independent manner.

Accordingly, compounds which are estrogen receptor modulators are useful in the treatment or inhibition of conditions, disorders, or disease states that are at least partially mediated by an estrogen deficiency or excess, or which may be treated or inhibited through the use of an estrogenic agent. Such compounds can be particularly useful in treating a peri-menopausal, menopausal, or postmenopausal patient in which the levels of endogenous estrogens produced are greatly diminished. For example, estrogenic compounds are also useful in inhibiting or treating hot flushes, vaginal or vulvar atrophy, atrophic vaginitis, vaginal dryness, pruritus, dyspareunia, dysuria, frequent urination, urinary incontinence, and urinary tract infections. Other reproductive tract uses include the treatment or inhibition of dysfunctional uterine bleeding and endometriosis.

Certain substituted benzoxazole compounds have been found to be effective estrogenic receptor modulators. An example of such a benzoxazole is 2-(3-fluoro-4-hydroxyphenyl)-7-vinyl-1,3-benzoxazol-5-ol, shown below in Formula I. The effectiveness of this compound as an estrogenic modulator, as well as its preparation, are reported in U.S. Pat. No. 6,794,403, which is hereby incorporated by reference in its entirety.

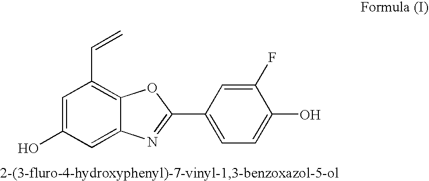

The crystal form of a particular drug (e.g., hydrate, solvate, polymorph, etc) is often an important determinant of the drug's ease of preparation, stability, solubility, storage stability, ease of formulation and in vivo pharmacology. Different crystal forms occur when a compound crystallizes in different lattice arrangements or where solvent molecules (including water molecules) are incorporated into the crystalline lattice, resulting in solids with different thermodynamic properties and stabilities specific to the particular form. It is entirely possible that one crystal form is preferable over another where certain aspects such as ease of preparation, stability, etc. are deemed to be critical. Similarly, greater solubility and/or superior pharmacokinetics may be the desired characteristics. An anhydrate crystal form and a monohydrate crystal form of the compound of Formula (I) have been reported in U.S. patent application Ser. No. 11/369,405, filed Mar. 6, 2006, which is hereby incorporated by reference in its entirety. Moreover, three additional different crystal forms of 2-(3-fluoro-4-hydroxyphenyl)-7-vinyl-1,3-benzoxazol-5-ol, three anhydrate crystal forms, have been disclosed in three U.S. provisional patent applications (U.S. provisional patent application Ser. No. 60/860,265 filed Nov. 21, 2006; U.S. provisional patent application Ser. No. 60/860,253 filed Nov. 21, 2006; and U.S. provisional patent application Ser. No. 60/860,246 filed Nov. 21, 2006), each of which is hereby incorporated by reference in its entirety.

Because improved drug formulations showing, for example, better bioavailability or better stability are consistently sought, there is an ongoing need for new or purer crystal forms of existing drug molecules. The crystal form of 2-(3-fluoro-4-hydroxyphenyl)-7-vinyl-1,3-benzoxazol-5-ol described herein are directed toward this and other important ends.

### SUMMARY OF THE INVENTION

The present invention provides a crystal form (designated as Form F herein) of 2-(3-fluoro-4-hydroxyphenyl)-7-vinyl-1,3-benzoxazol-5-ol, characterized according to the powder X-ray analysis provided herein.

The present invention further provides compositions containing the crystal form (Form F) of the invention.

The present invention further provides a method of preparing the crystal form (Form F) of the invention comprising precipitating the hydrate from an aqueous solution by adjusting the pH value of the solution.

The present invention further provides a method of preparing the crystal form (Form F) of the invention comprising precipitating the hydrate from a solution comprising a water-miscible organic solvent by adding water to the solution.

The present invention further provides compounds (the crystal form: Form F) prepared by the above methods.

The present invention further provides methods of modulating an estrogen receptor comprising contacting the receptor with the hydrate crystal form (Form F) of the Invention.

The present invention further provides methods of treating prostatitis, interstitial cystitis, inflammatory bowel disease, Crohn's disease, ulcerative proctitis, colitis, prostatic hypertrophy, uterine leiomyomas, breast cancer, endometrial cancer, polycystic ovary syndrome, endometrial polyps, endometriosis, benign breast disease, adenomyosis, ovarian cancer, melanoma, prostrate cancer, colon cancer, glioma, astioblastomia, free radical induced disease states, vaginal or vulvar atrophy, atrophic vaginitis, vaginal dryness, pruritus, dyspareunia, dysuria, frequent urination, urinary incontinence, urinary tract infections, vasomotor symptoms, arthritis, joint swelling or erosion, joint damage secondary to arthroscopic or surgical procedures, psoriasis, dermatitis, ischemia, reperfusion injury, asthma, pleurisy, multiple sclerosis, systemic lupus erythematosis, uveitis, sepsis, hemmorhagic shock, or type II diabetes, in a mammal in need thereof, which comprises providing to the mammal a therapeutically effective amount of the crystal form (Form F) of the invention.

The present invention further provides methods of lowering cholesterol, triglycerides, Lp(a), or LDL levels; inhibiting or treating hypercholesteremia, hyperlipidemia, cardiovascular disease, atherosclerosis, hypertension, peripheral vascular disease, restenosis, or vasospasm; or inhibiting vascular wall damage from cellular events leading toward immune mediated vascular damage in a mammal in need thereof, which comprises providing to the mammal a therapeutically effective amount of the crystal form (Form F) of the invention.

The present invention further provides methods of providing cognition enhancement or neuroprotection; or treating or inhibiting senile dementias, Alzheimer's disease, cognitive decline, stroke, anxiety, or neurodegenerative disorders in a mammal in need thereof, which comprises providing to the mammal an effective amount of the crystal form (Form F) of the invention.

The present invention further provides methods of inhibiting conception in a mammal in need thereof, which comprises providing to the mammal an effective amount of the crystal form (Form F) of the invention.

### BRIEF DESCRIPTION OF THE DRAWINGS

FIG. 1 depicts X-Ray powder diffraction (XRPD) pattern for the crystal form (Form F) of the invention.

### DETAILED DESCRIPTION

### Hydrate Crystal Form

The present invention provides, inter alia, a crystal form (designated as Form F herein) of 2-(3-fluoro-4-hydroxyphenyl)-7-vinyl-1,3-benzoxazol-5-ol (the compound of Formula (I)). The crystal form (Form F) of the compound of Formula (I) can be identified by its unique solid state signatures with respect to, for example, X-ray powder diffraction (XRPD), and other solid state methods. For XRPD, the relative intensities of the peaks can vary, depending upon the sample preparation technique, the sample mounting procedure and the particular instrument employed. Moreover, instrument variation and other factors can often affect the 2-theta values. Therefore, the peak assignments of diffraction patterns can vary by plus or minus about 0.2°. Accordingly, the term “about” as used in connection with a given 2-theta value, is intended to mean plus or minus 0.2°.

The X-ray data distinguishing the crystal form (Form F) of the invention is summarized in Table 1 below.

TABLE 1
X-ray data of the hydrate crystal form (Form F)

| Peak position, 2θ° | Peak Description |
|---|---|
| 5.8 | M |
| 10.0 | Strongest |
| 11.6 | S |
| 15.3 | S |
| 17.4 | W |
| 20.1 | M |
| 20.9 | VW |
| 23.2 | VW |
| 25.4 | W |
| 26.4 | VW |
| 29.2 | VW |

S: relatively high peak intensity
M: middle range peak intensity
W: relatively weak peak intensity
VW: very weak peak intensity

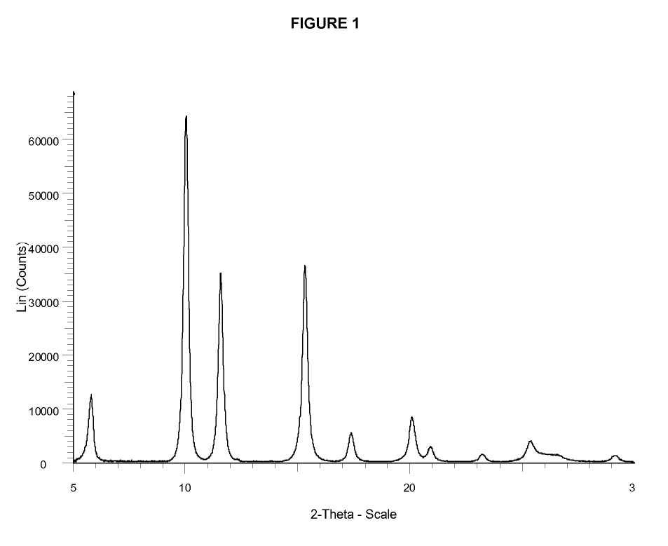

The crystal form (Form F) has a distinct XRPD pattern (see, e.g., FIG. 1), allowing characterization thereof based on the unique spectral signature. Accordingly, the present invention provides an crystal form of the compounds of Formula I having an X-ray powder diffraction pattern comprising peaks, in terms of 2θ, at about 10.0°, about 11.6°, and about 15.3°.

In some embodiments, the crystal form (Form F) has an X-ray powder diffraction pattern comprising peaks, in terms of 2θ, at about 5.8°, about 10.0°, about 11.6°, and about 15.3°. In some further embodiments, the crystal form has an X-ray powder diffraction pattern further comprising at least one peak, in terms of 2θ, selected from those at about 17.4°, about 20.1°, about 20.9°, about 23.2°, about 25.4°, about 26.4°, and about 29.2°. In some further embodiments, the crystal form has an X-ray powder diffraction pattern further comprising at least two peaks, in terms of 2θ, selected from those at about 17.4°, about 20.1°, about 20.9°, about 23.2°, about 25.4°, about 26.4°, and about 29.2°.

In some embodiments, the crystal form (Form F) has an X-ray powder diffraction pattern comprising peaks, in terms of 2θ, at about 5.8°, about 10.0°, about 11.6°, about 15.3°, about 17.4°, and about 20.1°. In some embodiments, the crystal form has an X-ray powder diffraction pattern comprising peaks, in terms of 2θ, at about 5.8°, about 10.0°, about 11.6°, about 15.3°, about 17.4°, about 20.1°, about 20.9°, about 23.2°, about 25.4°, about 26.4°, and about 29.2°. In further embodiments, the crystal form has an X-ray powder diffraction pattern substantially as shown in FIG. 1.

### Compositions

The present invention further provides compositions containing the crystal form (Form F) of the invention. In some embodiments, the compositions of the invention include at least about 50%, at least about 60%, at least about 70%, at least about 80%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, by weight of the crystal form (Form F) of the compound of Formula (I). In some embodiments, the compositions of the invention contain a mixture of the crystal form (Form F) and other crystal forms or amorphous forms of the compound of Formula (I). In some embodiments, compositions of the invention include the crystal form and a pharmaceutically acceptable carrier. In some embodiments, the compositions further include an additional active ingredient such as a progestin.

### Preparations

The crystal form (Form F) of the invention can be prepared by any of various suitable means. For example, the crystal form (Form F) can be prepared by precipitating the crystal form from a solution which comprises 2-(3-fluoro-4-hydroxyphenyl)-7-vinyl-1,3-benzoxazol-5-ol (the compound of Formula (I)). The compound of Formula (I) can be dissolved in an aqueous solution the pH of which is greater than about 9 (thus an alkaline solution). It is postulated that the compound of Formula (I) exists in the alkaline solution as an anion (one or both of the hydroxyl groups of the compound of Formula (I) can be deprotonated). In some embodiments, the pH value of the solution is adjusted to be between about 11 and about 12 in order to dissolve the compound of Formula (I) (thus to obtain a solution). The adjustment of the pH value of the solution can be carried out by adding a base, such as a metal hydroxide (e.g., NaOH, or KOH). The metal hydroxide can be in an aqueous solution to facilitate the addition.

The crystal form (Form F) of the invention can be precipitated out of the solution by adjusting the pH of the solution to a value of less than about 4.5 (thus the anion of the compound of Formula (I) is neutralized back to the compound of Formula (I), the crystal form of which does not have a significant solubility in water). In some embodiments, the adjustment of the pH value to less than about 4.5 is carried out by adding an inorganic acid such as hydrochloric acid or phosphoric acid. In some embodiments, the inorganic acid can be in an aqueous solution form to facilitate the addition. In some embodiments, the pH valuation of the solution is adjusted to be between about 2.5 and about 3.5 in order to precipitate the hydrate crystal form of the present invention. As used herein, the term “about” as used in connection with a given pH value, is intended to mean plus or minus 0.5.

Alternatively, the crystal form (Form F) can be precipitated from a solution of the compound of Formula (I) in a water-miscible organic solvent (i.e., an organic solvent miscible with water). Non-limiting examples of water miscible organic solvents include ketones (e.g., acetone or the like), alcohols (e.g., methanol, ethanol and isopropanol), organic nitrites (e.g., acetonitrile or the like), and mixtures thereof. In some embodiments, the hydrate is precipitated from a solution containing an alcohol, a ketone or an organic nitrile. In some embodiments, the hydrate is precipitated from a solution containing an alcohol or a ketone. In some embodiments, the crystal form is precipitated from a solution containing methanol, ethanol, isopropanol, acetone or acetonitrile. In some embodiments, the crystal form is precipitated from a solution containing methanol, ethanol, isopropanol or acetone. In some embodiments, the precipitation of the crystal form (Form F) of the invention is performed by adding water to the solution. In some such embodiments, the volume of the water added is at least one equivalent to that of the organic solvent, or between about two equivalents and three equivalents to that of the organic solvent in the solution. The crystal form precipitated out from the solution can be collected by any conventional methods such as filtration and optionally be dried at room temperature (optionally under vacuum).

Not wishing to be bound by any particular theory, it is believed that the crystal form (Form F) is a hydrate crystal form of the compound of Formula (I), and that the hydrate crystal form can contain various amount of water (for example, the molar ratio of the hydrate water to the compound of Formula (I) in the crystal form can be about 1:1, about 2:1, about 3:1, or about 4:1). It is further understood that the crystal form (or the hydrate crystal form) of the invention is stable at an environment where water (or moisture) is present. The crystal form (or the hydrate crystal form) of the present invention (Form F) can further be dried (at an elevated temperature such as about 50° C.) to obtain one or more of anhydrate crystal forms, amorphous form, and/or other hydrate crystal forms.

### Methods of Use and Pharmaceutical Formulations

The crystal form (Form F) of this invention is an estrogen receptor modulator useful in the treatment or inhibition of conditions, disorders, or disease states that are at least partially mediated by an estrogen deficiency or excess, or which can be treated or inhibited through the use of an estrogenic agent. Accordingly, the present invention provides a method of modulating an estrogen receptor comprising contacting the receptor with a hydrate crystal form of the invention. The hydrate crystal form of this invention is particularly useful in treating a peri-menopausal, menopausal, or postmenopausal patient in which the levels of endogenous estrogens produced are greatly diminished. Menopause is generally defined as the last natural menstrual period and is characterized by the cessation of ovarian function, leading to the substantial diminution of circulating estrogen in the bloodstream. As used herein, menopause also includes conditions of decreased estrogen production that may be surgically, chemically, or be caused by a disease state which leads to premature diminution or cessation of ovarian function.

The crystal form (Form F) of this invention is also useful in inhibiting or treating other effects of estrogen deprivation including, hot flushes, vaginal or vulvar atrophy, atrophic vaginitis, vaginal dryness, pruritus, dyspareunia, dysuria, frequent urination, urinary incontinence, and urinary tract infections. Other reproductive tract uses include the treatment or inhibition of dysfunctional uterine bleeding. The crystal form is also useful in treating or inhibiting endometriosis.

The crystal form (Form F) of this invention is also active in the brain and are therefore useful for inhibiting or treating Alzheimer's disease, cognitive decline, decreased libido, senile dementia, neurodegenerative disorders, depression, anxiety, insomnia, schizophrenia, and infertility. The crystal form (Form F) of this invention is also useful in treating or inhibiting benign or malignant abnormal tissue growth including, glomerulosclerosis, prostatic hypertrophy, uterine leiomyomas, breast cancer, scleroderma, fibromatosis, endometrial cancer, polycystic ovary syndrome, endometrial polyps, benign breast disease, adenomyosis, ovarian cancer, melanoma, prostate cancer, cancers of the colon, and CNS cancers, such as glioma or astioblastomia.

The crystal form (Form F) of this invention is cardioprotective and is antioxidants, and is useful in lowering cholesterol, triglycerides, Lp(a), and LDL levels; inhibiting or treating hypercholesteremia, hyperlipidemia, cardiovascular disease, atherosclerosis, peripheral vascular disease, restenosis, and vasospasm; and inhibiting vascular wall damage from cellular events leading toward immune mediated vascular damage. The compound of this invention is also useful in treating disorders associated with inflammation or autoimmune diseases, including inflammatory bowel disease (Crohn's disease, ulcerative colitis, indeterminate colitis), arthritis (rheumatoid arthritis, spondyloarthropathies, osteoarthritis), pleurisy, ischemia/reperfusion injury (e.g. stroke, transplant rejection, myocardial infarction, etc.), asthma, giant cell arteritis, prostatitis, uveitis, psoriasis, multiple sclerosis, systemic lupus erythematosus and sepsis.

The crystal form (Form F) of this invention is also useful in treating or inhibiting ocular disorders, including cataracts, uveitis, and macular degeneration, and in treating skin conditions such as aging, alopecia, and acne.

The crystal form (Form F) of this invention is also useful in treating or inhibiting metabolic disorders such as type-II diabetes, of lipid metabolism, and of appetite (e.g. anorexia nervosa and bulimia).

The crystal form (Form F) of this invention is also useful in treating or inhibiting bleeding disorders such as hereditary hemorrhagic telangiectasia, dysfunctional uterine bleeding, and combating hemorrhagic shock.

The crystal form (Form F) of this invention is useful in disease states where amenorrhea is advantageous, such as leukemia, endometrial ablations, chronic renal or hepatic disease or coagulation diseases or disorders.

The crystal form (Form F) of this invention can be used as a contraceptive agent, particularly when combined with a progestin.

Methods of treating the diseases and syndromes listed herein are understood to involve administering to an individual in need of such treatment a therapeutically effective amount of a crystal form of the invention, or composition containing the same. As used herein, the term “treating” in reference to a disease is meant to refer to preventing, inhibiting and/or ameliorating the disease.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

As used herein, the term “contacting” refers to the bringing together of indicated moieties in an in vitro system or an in vivo system. For example, “contacting” an estrogen receptor with a crystal form of the invention includes the administration of a crystal form of the present invention to an individual or patient, such as a human, having an estrogen receptor, as well as, for example, introducing a crystal form of the invention into a sample containing a cellular or purified preparation containing the estrogen receptor.

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. Effective administration of the crystal forms of this invention may be given at an oral dose of from about 0.1 mg/day to about 1,000 mg/day. Preferably, administration will be from about 10 mg/day to about 600 mg/day, more preferably from about 50 mg/day to about 600 mg/day, in a single dose or in two or more divided doses. The projected daily dosages are expected to vary with route of administration.

Such doses may be administered in any manner useful in directing the active compounds herein to the recipient's bloodstream, including orally, via implants, parentally (including intravenous, intraperitoneal, intraarticularly and subcutaneous injections), rectally, intranasally, topically, ocularly (via eye drops), vaginally, and transdermally.

Oral formulations containing the active crystal forms of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the crystal form directly to the airways in the form of an aerosol.

The crystal form (Form F) of this invention may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

In order that the invention disclosed herein may be more efficiently understood, examples are provided below. It should be understood that these examples are for illustrative purposes only and are not to be construed as limiting the invention in any manner.

### EXAMPLES

### Example 1

### Preparation of the Crystal Form

### Form F (Preparation 1)

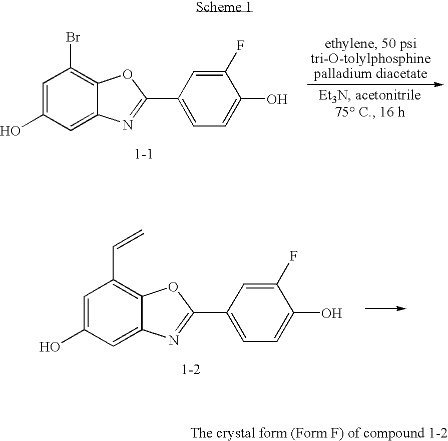

According to Scheme 1, a 2-gallon hydrogenator was charged with 2-(3-Fluoro-4-hydroxyphenyl)-7-bromobenzooxazol-5-ol (compound 1-1, 280.0 g, 0.926 mole), tri-o-tolylphosphine (8.49 g, 3.3%), palladium diacetate (1.96 g, 1%), acetonitrile (3.36 L), triethylamine (350 g, 4.0 eq). The hydrogenator is flushed with nitrogen, and with ethylene; then the pressure is adjusted to 50 psi and heated to 75° C. and held for 16 h. HPLC analysis revealed that only 0.2% starting material (compound 1-1) remained in the reaction mixture. The mixture was cooled to 35-40° C. and filtered through a 0.2μ cartridge, washed with acetonitrile (0.66 L) at 60-70° C. One third (33% or 0.288 mole, which is equivalent to 93.33 g of starting material) of the filtrate was concentrated under vacuum to 0.4 L, and added water (0.4 L) and tert-butyl-methylether (TBME, 0.3 L). Then the pH of the aqueous layer was adjusted to 11-12 by adding at 15-20° C. 30% NaOH aqueous solution (0.063 kg). The phases were separated and the organic phase was extracted with water (0.09 L), and 30% NaOH (5 grams). The combined aqueous phase is washed with TBME (2×0.3 L); then the pH of the aqueous layer was adjusted to 2.5-3.5 by adding at 15-20° C. 10% HCl aqueous solution (100 g) and held for 2 h. The solid was filtered off from the mixture and washed with water (2×0.07 L). The cake of the filtered solid was suspended in water (1 L) for 1 h and the solid (the crystal form, i.e., Form F) was filtered off and washed with water (3×0.07 L).

### Example 2

### Acquisition of X-Ray Powder Diffraction Data of Form F

X-Ray data (see, e.g., Table 1) was acquired using an X-ray powder diffractometer (Bruker-axs, model D8 advance, Vantec-1 detector) having the following parameters: voltage 40 kV, current 40.0 mA, scan range (2θ) 6 to 30°, scan step size 0.01°, total scan time 29 minutes with 1 mm divergence slit and a Ni filter.

### Example 3

### Preparation of a Pharmaceutical Formulation and Composition Containing the Hydrate Crystal Form (Form F) of the Invention (Unit Dose of 75 Mg/Tablet)

The pharmaceutical formulation is prepared by steps 1-7 of the following procedure utilizing the weight/weight percentages (% wt/wt) of the ingredients shown in the table below. The tablets are prepared by steps 8-10 of the following procedure. Each tablet contained the unit dose amounts shown in the table below.

1. An aqueous solution of polyvinylpyrrolidone (povidone K25) and sodium lauryl sulfate in purified water is prepared.
2. The crystal form (Form F) of 2-(3-fluoro-4-hydroxyphenyl)-7-vinyl-1,3-benzoxazol-5-ol is mixed with a portion of mannitol (Pearlitol 200SD), and then the mixture is passed through an appropriate screen and placed in a high shear mixer bowl.
3. The remainder of the mannitol, microcrystalline cellulose (Avicel pH 113), and croscarmellose sodium is passed through an appropriate screen into the mixer bowl and mixed.
4. The blend from Step 3 is granulated using the Step 1 solution, and is followed with additional purified water if needed.
5. The Step 4 granulation is dried and passed through an appropriate screen.
6. The magnesium stearate is passed through an appropriate screen.
7. The magnesium stearate is premixed with an equal portion of the blend in Step 5, and then the premix is added to the remainder of the Step 5 material and mixed in a blender.
8. The final blend from Step 7 is compressed into tablets using a suitable tablet press.
9. A 7.5% solid solution of Opaglos 2 is prepared.
10. A sufficient amount of coating solution is applied on the tablets to provide a 3.0% wt/wt increase in dried tablet weight.

Composition of the Pharmaceutical Formulation and Tablet

| | % wt/wt in the | |
|---|---|---|
| | pharmaceutical | Unit Dose |
| Ingredient | formulation batch | (mg/tablet) |
| The Crystal Form (Form F) of 2-(3- | 25.0 | 75.0 |
| fluoro-4-hydroxyphenyl)-7-vinyl-1,3- | | |
| benzoxazol-5-ol | | |
| Mannitol (Pearlitol 200SD) ^{a} | 51.5 | 154.5 |
| Microystalline Cellulose (Avicel PH | 15.0 | 45.0 |
| 113) | | |
| Croscarmellose Sodium | 4.0 | 12.0 |
| Polyvinylpyrrolidone (Povidone K25) | 2.0 | 6.0 |
| Sodium Lauryl Sulfate | 2.0 | 6.0 |
| Magnesium Stearate | 0.5 | 1.5 |
| Purified Water ^{b} | — | — |
| Total | 100.0% | 300.0 |
| Film Coat | 3.0 | 9.0 |
| Opaglos 2, green | | |
| 97W11753 | | |

^{a} If assay is other than 100.0%, adjust the amount of input against mannitol accordingly.
^{b} Used in the process, but does not appear in the final tablet product.

### Example 4

### Preparation of a Pharmaceutical Formulation and Composition Containing the Hydrate Crystal Form (Form F) of the Invention (Tablet Unit Dose of 25 Mg/Tablet)

The pharmaceutical formulation is prepared by steps 1-7 of the procedure of Example 3, utilizing the weight/weight percentages (% wt/wt) of the ingredients shown in the table below. The tablets are prepared by steps 8-10 of the procedure of Example 3. Each tablet contained the unit dose amounts shown in the table below.

Composition of the Tablet:

| | % wt/wt in the | |
|---|---|---|
| | pharmaceutical | Unit Dose |
| Ingredient | formulation batch | (mg/tablet) |
| The Crystal Form (Form F) of 2-(3- | 25.0 | 25.0 |
| fluoro-4-hydroxyphenyl)-7-vinyl-1,3- | | |
| benzoxazol-5-ol | | |
| Mannitol (Pearlitol 200SD) ^{a} | 51.5 | 51.5 |
| Microystalline Cellulose (Avicel PH | 15.0 | 15.0 |
| 113) | | |
| Croscarmellose Sodium | 4.0 | 4.0 |
| Polyvinylpyrrolidone (Povidone K25) | 2.0 | 2.0 |
| Sodium Lauryl Sulfate | 2.0 | 2.0 |
| Magnesium Stearate | 0.5 | 0.5 |
| Purified Water ^{b} | — | — |
| Total | 100.0% | 100.0 |
| Film Coat | 3.0 | 3.0 |
| Opaglos 2, green | | |
| 97W11753 | | |

^{a} If assay is other than 100.0%, adjust the amount of input against mannitol accordingly.
^{b} Used in the process, but does not appear in the final tablet product.

### Example 5

### Preparation of a Pharmaceutical Formulation and Composition Containing the Crystal form (Form F) of the Invention (Tablet Unit Dose of 5 Mg/Tablet)

The pharmaceutical formulation is prepared by steps 1-7 of the procedure of Example 3, utilizing the weight/weight percentages (% wt/wt) of the ingredients shown in the table below. The tablets are prepared by steps 8-10 of the procedure of Example 3. Each tablet contained the unit dose amounts shown in the table below.

Composition of the Tablet:

| | % wt/wt in the | |
|---|---|---|
| | pharmaceutical | Unit Dose |
| Ingredient | formulation batch | (mg/tablet) |
| The Crystal Form (Form F) of 2-(3- | 5.0 | 5.0 |
| fluoro-4-hydroxyphenyl)-7-vinyl-1,3- | | |
| benzoxazol-5-ol | | |
| Mannitol (Pearlitol 200SD) ^{a} | 71.5 | 71.5 |
| Microystalline Cellulose (Avicel PH | 15.0 | 15.0 |
| 113) | | |
| Croscarmellose Sodium | 4.0 | 4.0 |
| Polyvinylpyrrolidone (Povidone K25) | 2.0 | 2.0 |
| Sodium Lauryl Sulfate | 2.0 | 2.0 |
| Magnesium Stearate | 0.5 | 0.5 |
| Purified Water ^{b} | — | — |
| Total | 100.0% | 300.0 |
| Film Coat | 3.0 | 3.0 |
| Opaglos 2, green | | |
| 97W11753 | | |

^{a} If assay is other than 100.0%, adjust the amount of input against mannitol accordingly.
^{b} Used in the process, but does not appear in the final tablet product.

### Example 6

### Preparation of a Pharmaceutical Formulation and Composition Containing the Crystal form (Form F) of the Invention (Tablet Unit Dose of 150 Mg/Tablet)

The pharmaceutical formulation is prepared by steps 1-7 of the procedure of Example 3, utilizing the weight/weight percentages (% wt/wt) of the ingredients shown in the table below. The tablets are prepared by steps 8-10 of the procedure of Example 3. Each tablet contained the unit dose amounts shown in the table below.

Composition of the Tablet:

| | % wt/wt in the | |
|---|---|---|
| | pharmaceutical | Unit Dose |
| Ingredient | formulation batch | (mg/tablet) |
| The Crystal Form (Form F) of 2-(3- | 25.0 | 150.0 |
| fluoro-4-hydroxyphenyl)-7-vinyl-1,3- | | |
| benzoxazol-5-ol | | |
| Mannitol (Pearlitol 200SD) ^{a} | 51.5 | 309.0 |
| Microystalline Cellulose (Avicel PH | 15.0 | 90.0 |
| 113) | | |
| Croscarmellose Sodium | 4.0 | 24.0 |
| Polyvinylpyrrolidone (Povidone K25) | 2.0 | 12.0 |
| Sodium Lauryl Sulfate | 2.0 | 12.0 |
| Magnesium Stearate | 0.5 | 3.0 |
| Purified Water ^{b} | — | — |
| Total | 100.0% | 600.0 |
| Film Coat | 3.0 | 18.0 |
| Opaglos 2, green | | |
| 97W11753 | | |

^{a} If assay is other than 100.0%, adjust the amount of input against mannitol accordingly.
^{b} Used in the process, but does not appear in the final tablet product.

Various modifications of the invention, in addition to those described herein, will be apparent to those skilled in the art from the foregoing description. Such modifications are also intended to fall within the scope of the appended claims. Each reference cited in the present application, including patents, published applications, and journal articles, is hereby incorporated by reference in its entirety.

What is claimed is:

1. A crystal form (Form F) of 2-(3-fluoro-4-hydroxyphenyl)-7-vinyl-1,3-benzoxazol-5-ol having an X-ray powder diffraction pattern comprising peaks, in terms of 2θ, at about 10.0°, about 11.6°, and about 15.3°.

2. The crystal form of claim 1 having an X-ray powder diffraction pattern comprising peaks, in terms of 2θ, at about 5.8°, about 10.0°, about 11.6°, and about 15.3°.

3. The crystal form of claim 2 wherein the X-ray powder diffraction pattern further comprises at least one peak, in terms of 2θ, selected from those at about 17.4°, about 20.1°, about 20.9°, about 23.2°, about 25.4°, about 26.4°, and about 29.2°.

4. The crystal form of claim 2 wherein the X-ray powder diffraction pattern further comprises at least two peaks, in terms of 2θ, selected from those at about 17.4°, about 20.1°, about 20.9°, about 23.2°, about 25.4°, about 26.4°, and about 29.2°.

5. The crystal form of claim 1 having an X-ray powder diffraction pattern comprising peaks, in terms of 2θ, at about 5.8°, about 10.0°, about 11.6°, about 15.3°, about 17.4°, and about 20.1°.

6. The crystal form of claim 1 having an X-ray powder diffraction pattern comprising peaks, in terms of 2θ, at about 5.8°, about 10.0°, about 11.6°, about 15.3°, about 17.4°, about 20.1°, about 20.9°, about 23.2°, about 25.4°, about 26.4°, and about 29.2°.

7. The crystal form of claim 1 having an X-ray powder diffraction pattern substantially as shown in FIG. 1.

8. A composition comprising the crystal form of claim 1.

9. The composition of claim 8 wherein said crystal form constitutes at least about 50% by weight of said composition.

10. The composition of claim 8 wherein said crystal form constitutes at least about 80% by weight of said composition.

11. The composition of claim 8 wherein said crystal form constitutes at least about 90% by weight of said composition.

12. The composition of claim 8 wherein said crystal form constitutes at least about 95% by weight of said composition.

13. The composition of claim 8 wherein said crystal form constitutes at least about 98% by weight of said composition.

14. The composition of claim 8 wherein said crystal form constitutes at least about 99% by weight of said composition.

15. The composition of claim 8 wherein said crystal form constitutes at least about 99.5% by weight of said composition.

16. The composition of claim 8 wherein said crystal form constitutes at least about 99.9% by weight of said composition.

17. A composition comprising the crystal form of claim 1 and a pharmaceutically acceptable carrier.

18. A process for preparing the crystal form of claim 1 comprising precipitating said crystal form from a solution which comprises 2-(3-fluoro-4-hydroxyphenyl)-7-vinyl-1,3-benzoxazol-5-ol.

19. The process of claim 18 wherein said solution comprises water and the pH value of the solution is greater than about 9.

20. The process of claim 19 wherein said solution comprises water and the pH value of the solution is from about 11 to about 12.

21. The process of claim 19 wherein said solution comprises water and an inorganic base.

22. The process of claim 19 wherein said solution comprises water and sodium hydroxide.

23. The process of claim 19 wherein said precipitating comprises adjusting the pH value of the solution to less than about 4.5.

24. The process of claim 19 wherein said precipitating comprises adjusting the pH value of the solution to a value of from about 2.5 to about 3.5.

25. The process of claim 19 wherein said adjusting the pH value of the solution comprises adding an inorganic acid to the solution.

26. The process of claim 19 wherein said adjusting the pH value of the solution comprises adding hydrochloric acid to the solution.

27. The process of claim 18 wherein said solution comprises a water-miscible organic solvent; and wherein the precipitating comprises adding water to the solution wherein the volume of the water is at least one equivalent to that of the organic solvent.

28. The process of claim 18 wherein said solution comprises a water-miscible organic solvent; and wherein the precipitating is carried out by adding water to the solution wherein the volume of the water is about two to three equivalents to that of the organic solvent.

29. A crystal form (Form F) of 2-(3-fluoro-4-hydroxyphenyl)-7-vinyl-1,3-benzoxazol-5-ol prepared by the process of claim 21.

30. A method of modulating an estrogen receptor comprising contacting said receptor with the crystal form of claim 1.

31. A method of treating prostatitis, interstitial cystitis, inflammatory bowel disease, Crohn's disease, ulcerative proctitis, colitis, prostatic hypertrophy, uterine leiomyomas, breast cancer, endometrial cancer, polycystic ovary syndrome, endometrial polyps, endometriosis, benign breast disease, adenomyosis, ovarian cancer, melanoma, prostrate cancer, colon cancer, glioma, astioblastomia, free radical induced disease states, vaginal or vulvar atrophy, atrophic vaginitis, vaginal dryness, pruritus, dyspareunia, dysuria, frequent urination, urinary incontinence, urinary tract infections, vasomotor symptoms, arthritis, joint swelling or erosion, joint damage secondary to arthroscopic or surgical procedures, psoriasis, dermatitis, ischemia, reperfusion injury, asthma, pleurisy, multiple sclerosis, systemic lupus erythematosis, uveitis, sepsis, hemmorhagic shock, or type II diabetes, in a mammal in need thereof, which comprises providing to said mammal a therapeutically effective amount of the crystal form of claim 1.

32. A method of lowering cholesterol, triglycerides, Lp(a), or LDL levels; inhibiting or treating hypercholesteremia, hyperlipidemia, cardiovascular disease, atherosclerosis, hypertension, peripheral vascular disease, restenosis, or vasospasm; or inhibiting vascular wall damage from cellular events leading toward immune mediated vascular damage in a mammal in need thereof, which comprises providing to said mammal a therapeutically effective amount of the crystal form of claim 1.

33. A method of providing cognition enhancement or neuroprotection; or treating or inhibiting senile dementias, Alzheimer's disease, cognitive decline, stroke, anxiety, or neurodegenerative disorders in a mammal in need thereof, which comprises providing to said mammal an effective amount of the crystal form of claim 1.

34. A method of inhibiting conception in a mammal in need thereof, which comprises providing to said mammal an effective amount of the crystal form of claim 1.

35. The crystal form of claim 1 wherein the crystal form comprises hydrate water.
